(19) United States
(12) Patent Application Publication
(10) Pub. No.: US 2014/0275942 A1
(43) Pub. Date: Sep. 18, 2014

(54) Imaging Device for Biomedical Use

(71) Applicant: BOISE STATEMENT UNIVERSITY, Boise, ID (US)

(72) Inventors: Jami Johnson, Boise, ID (US); Kasper VanWijk, Boise, ID (US)

(21) Appl. No.: 13/838,359

(22) Filed: Mar. 15, 2013

(51) Int. Cl. A61B 5/00

(52) U.S. Cl. CPC A61B 5/0095
USPC 600/407

(57) ABSTRACT

An imaging system that includes a light source configured to project a beam of light within a determined range of wavelengths onto an illumination area of a patient's skin and a detector configured to detect acoustic waves within a scan area of the patient's skin without contacting the patient's skin, wherein the scan area does not overlap the illumination area. In certain embodiments, the beam of light is a series of pulses of coherent light and the detector is an interferometer.

### STATEMENT REGARDING FEDERALLY SPONSORED RESEARCH OR DEVELOPMENT

This invention was made with government support under one or more of SBAHQ-09-I-0113 and SBAHQ-11-I-0115 awarded by the Small Business Administration and EAR-1229722 awarded by the National Science Foundation. The government has certain rights in the invention.

### BACKGROUND

The present invention generally relates to imaging systems and, in particular, systems using optical stimulation to generate acoustic responses that are analyzed to generate an image of structures within a body.

Systems that operate non-invasively to provide images of structures within a patient's body are a valuable tool in many areas of healthcare. There are a number of technologies that may be used to generate two-dimensional or three-dimensional images, depending on the structure to be imaged.

Ultrasonic imaging systems, or sonography systems, are suited for viewing soft tissue structures and are commonly used to view the fetus of a pregnant woman. Sonography operates by sensing the reflection of ultrasonic waves generated by a piezoelectric transducer held against the skin. Ultrasonic systems can provide images to a depth of 5 cm or deeper with limited resolution and are suited to detect internal organs with relatively dense surfaces.

Infrared viewing systems exist that project infrared onto a patient's skin and optically observe arteries up to 10 mm deep, due to the preferential absorption of the infrared light by hemoglobin. The systems may then project a two-dimensional visible-light image of the arteries onto the patient's skin to guide the caregiver, for example in placing a needle into an artery.

Thermoacoustic tomography (TAT) systems use microwaves to heat internal structures, such as arteries, thereby generating pressure (acoustic) waves that travel to the skin surface where the waves may be detected and analyzed to develop an image of the internal structure. As the microwaves are absorbed by water, which constitutes a major portion of most tissue, the resolution and depth are limited.

Photoacoustic imaging (PAI) systems project light that penetrates into the tissue below the skin and heats internal structures, such as arteries, due to absorption of the light by substances such as hemoglobin, lipids, and melanin in the structure. The absorbed heat causes a momentary tissue expansion thereby generating acoustic waves that travel to the skin surface where the waves may be detected by, in conventional systems, sensors in contact with the skin and analyzed to develop an image of the internal structure.

### SUMMARY OF THE DISCLOSURE

It is desirable to provide a non-contact system that provides high-resolution images of arteries and veins and substances within them.

In certain embodiments, an imaging system is disclosed that includes a light source configured to project a beam of light within a determined range of wavelengths onto an illumination area of a patient's skin and a detector configured to detect acoustic waves within a scan area of the patient's skin without contacting the patient's skin, wherein the scan area does not overlap the illumination area.

In certain embodiments, a method of creating an image of a structure below a patient's skin is disclosed. The method includes the steps of illuminating an illumination area of the patient's skin with a pulse of light, detecting the arrival of acoustic waves at the patient's skin within a scan area that does not overlap the illumination area, and analyzing the detected acoustic waves to create an image of the structure.

The features of the present disclosure will be readily apparent to those skilled in the art upon a reading of the description of the embodiments that follows.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIGS. 1A-1D schematically depict the process of imaging a subdermal artery according to certain aspects of the present disclosure.

FIG. 2 displays a representation of acoustic waves within tissue according to certain aspects of the present disclosure.

FIG. 3 is a plot illustrative of the absorption curves for water, hemoglobin (Hb), and oxyhemoglobin (HbO2).

FIGS. 4-6 are plots of acoustic signals for simulated arteries at depths of 13, 20, and 34 mm according to certain aspects of the present disclosure.

FIG. 7 is a perspective image of an exemplary imaging system according to certain aspects of the present disclosure.

FIGS. 8-11 depict example illumination and scan areas according to certain aspects of the present disclosure.

### DETAILED DESCRIPTION

The following description discloses embodiments of an imaging device that uses photoacoustic stimulation and laser-ultrasound detection to generate images of internal structures, such as arteries, within the body of a patient. In certain embodiments, this type of imaging system may be used as a diagnostic aid or to guide insertion of a catheter into an artery or a biopsy needle into a subdermal mass.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Components may be referred to as a general item with a reference identifier without a suffix, for example “126,” while replicates of the same component may be individually identified with the same reference identifier with a suffix, for example “126A,” “126B,” and “126C.”

FIGS. 1A-1D depict the process of imaging a subdermal artery 30 with an imaging system 100, according to certain aspects of the present disclosure. The artery 30 is situated within tissue 20 and below the skin 25. The imaging system 100 includes a laser source 110 and a laser detector 130. FIG. 1A shows the system 100 at a time T₀ while the laser source 110 projecting an illumination beam 112 that, in certain embodiments, is a series of pulses of coherent light having a pulse duration that is within a determined range of durations and a pulse frequency that is within a determined range of wavelengths. As illustrated, the illuminating beam 112 penetrates the tissue 20 and irradiates the artery 30. In this example, the illumination beam 112 is provided along an illumination axis 114 arranged at an angle so as to intersect a reference zone, indicated by the region between the reference lines 136, that is defined as the area scanned by the laser detector 130 projected perpendicular to the skin 25. In certain embodiments, the target subdermal structure may be at a depth of 0-60 mm. In certain embodiments, the target subdermal structure may be at a depth of 5-30 mm. In certain embodiments, the target subdermal structure may be at a depth of 10-20 mm. The laser detector 130 in this example is emitting a detector beam 132 that is scanning the skin 25 within a solid angle 134. In certain embodiments, other types of laser detectors known to those of skill in the art that scan perpendicular to the skin 25 may be used.

FIG. 1B illustrates the system 100 at time T₁, subsequent to T₀, and after the laser source 110 has stopped emitting the laser pulse 112. A portion of the energy of the pulse 112 was absorbed by the artery 30 and converted into heat, causing a short-lived temperature rise in the artery 30 and any fluid disposed within the artery 30, thereby causing a local pressure increase that expands the artery 30 to the configuration 31. The actual amount of the expansion is extremely small. The original configuration, i.e. size, of the artery 30 is shown in FIG. 1B by the dashed-line ellipse labeled 30′ and the artery 30 very quickly returns to this original configuration. This thermoelastic expansion emits an acoustic wave 140, referred to as a photoacoustic (PA) wave. The skin 25 also absorbs a portion of the energy of the pulse 112, if the wavelength of the source is above about 1000 nm, and emits a pressure wave 150, referred to as a laser ultrasound (LU) wave, that travels through the tissue 20 and away from the skin 25. The structure of the PA and LU waves 140, 150 are discussed in greater detail with respect to FIG. 2.

FIG. 1C is the configuration at time T₂, subsequent to time T₁, and after the PA wave 140 has reached the skin 25 and the LU wave 150 has reached the artery 30. The detector beam 132 measures the displacement and two-dimensional location of the displacement of the skin caused by the PA wave 140 as it reaches the skin 25, thereby mapping the displacement at a particular instant in time. The LU beam 150 was partially reflected by the artery 30 to create a reflected LU wave 155 that travels back toward the skin 25.

FIG. 1D shows the configuration at time T₃, subsequent to time T₂, and after the center of the PA wave 140 has passed beyond the skin 25 and the PA wave 140 is now causing an expanding ring 141 of skin 25 to be displaced. The ring 141 may be circular or elliptical or have other shapes that depend on the location and depth of the artery 30. The detector beam 132 continues to map the height or displacement of the skin 25 within the scanned region 134 at time intervals that are short enough that the shape and location of the expanding ring 141 of displaced skin 25 are mapped multiple times. The LU wave 155 continues to travel towards the skin 25. When the LU wave 155 arrives at the skin 25, it will cause motion or displacement of the skin 25 generally in the same manner as the PA wave 140 and the deflection will be measured by the laser detector 130.

FIG. 2 displays a representation of acoustic waves 140, 155 within the tissue 25, according to certain aspects of the present disclosure. The references identified in FIG. 2 are copied from similar PA and LU waves in FIGS. 1A-1D to aid in the present explanation. The plot 200 of FIG. 2 is illustrative in nature and may not necessarily correspond to the dimensions and arrangement of the features of FIGS. 1A-1D, and therefore should not be considered as limiting the present disclosure.

With continued reference to FIG. 1A, the vertical y-axis of FIG. 2 represents a single dimension of the area of skin 25 that is scanned by the detector beam 132 and the distance is measured from an arbitrary edge of the scanned area. The horizontal x-axis of FIG. 2 represents the time of arrival of the waves 140, 155 at the distance at the same height on the y-axis. The plot 200 of FIG. 2 is thus equivalent to a snapshot of the location of the waves 140, 155 within the tissue 20 at a particular instant in time with the skin 25 is positioned on the vertical axis and the depth of the waves 140, 155 below that skin 25 are linearly related to the time of the x-axis, with greater time equivalent to greater depth.

Portions of the background of the plot 200 are shaded to indicate a zero velocity, with positive velocity being defined as toward the skin 25, i.e. to the left in the plot 200. Representative velocities are shown in the legend of plot 200, wherein a positive velocity of 6×10⁻⁴ meters/second (m/s) has a very dark shading and a negative velocity of −9×10⁻⁴ m/s is relatively un-shaded (i.e., white). The PA wave 140 has region 140P having a positive velocity, shown as a darker shade, that is followed by a region 140N of negative velocity, shown as a shade that is lighter than the background. The LU wave 155, however, has a leading portion 155N with a negative velocity that is followed by a positive-velocity portion 155P.

The waves 140, 155 are moving toward the skin 25, i.e. to the left in plot 200. It can be seen that the PA wave 140 will reach the skin 25 and be detected by the laser detector 130 first, followed by the reflected LU wave 155 after a time interval, whereupon the reflected LU wave 155 will be detected by the same laser detector 130. The LU wave 155 will arrive later, and is located at the observed instant in time at a deeper location, due to the origination of the LU wave 150, as shown in FIG. 1B, at the skin 25 and having to travel from the skin 25 to the subdermal structure, e.g. the artery 30, to generate the reflected LU wave 155 whereas the PA wave 140 originated directly at the subdermal structure. The combination and analysis of the series of mapped displacement of the skin 25 in order to generate an image of the internal structure, e.g. the artery 30, that created the PA wave 140 and the reflected LU wave 155 are known to those of skill in the art and are not repeated herein.

FIG. 3 is a plot 300 illustrative of the absorption curves for water, hemoglobin (Hb), and oxyhemoglobin (HbO₂). Curve 310 is water, curve 320 is Hb, and curve 325 is HbO₂. Since arterial blood is almost completely oxygenated, the HbO2 curve 325 is representative of the characteristics of an artery. Selection of a stimulation laser frequency that is poorly absorbed by water and having a relatively high absorption by arterial blood, for example the range of 700-900 nm, may provide the optimal transfer of energy to an artery to generate a PA wave. However, light having a wavelength of 1000 nm or greater will couple better to the skin to create a stronger LU wave while still creating PA waves. Thus, in certain embodiments, the illuminating beam may be in the range of 500-2000 nm and the selection of the wavelength of light for the illuminating light, e.g., the laser pulse 112, may depend on whether it is desired to utilize one or both of a PA wave and an LU wave to generate an image. In certain embodiments, illuminating beam may have a frequency in the 1000-1200 nm range. In certain embodiments, the illuminating beam may be in the range of 1050-1150 nm. In certain embodiments, the illuminating beam may have a frequency of approximately 1064 nm.

FIGS. 4-6 are plots of acoustic signals for simulated arteries at depths of 13, 20, and 34 mm, respectively, according to certain aspects of the present disclosure. A solid “phantom” that simulates the relevant characteristics of tissue was created using de-ionized water, 1.2% INTRALIPID®, and 1% agar. Three thin-walled polyester tubes, simulating arteries, were placed at depths of 13, 20, and 34 mm below the surface of the phantom. A 1064 nm Nd:YAG laser was used as an excitation source with a 15 nanosecond pulse to illuminate a backside of the phantom. The acoustic waves were detected on a front side of the phantom with a scanning interferometer. The tube to be tested was filled with a dye that absorbs 1064 nm light. The absorbing dye in the tubes is representative of a substance in the body that has unique absorption spectra, such as hemoglobin or lipids. It should be understood that, although this experiment is arranged in a “transmissive” configuration, the signals are illustrative of signals produced by PA and LU waves in a “reflective” configuration and are provided herein to further illustrate the fundamental principles and do not limit the scope of the disclosure.

FIG. 4 depicts the magnitude of the PA waves that are detected at a single point on the surface of the phantom for the simulated artery at a depth of 34 mm. The first PA wave 402 arrives approximately 22 microseconds after the laser pulse, with the positive velocity portion of the wave preceding the negative velocity portion of the wave. The distortion of the continuing oscillations at the point 404 just after 30 microseconds indicates the arrival of the first LU wave 404.

The plot 410 of FIG. 5 shows the detected waves for the simulated artery that is a 20 mm depth. The first PA wave 412 arrives at approximately 16 microseconds, followed by a reflected LU wave 414 at approximately 28 microseconds. The PA wave 412 arrives sooner than the equivalent PA wave 402 from FIG. 4 because the distance traveled by the PA wave 412 is shorter, being 20 mm versus 34 mm. The LU wave 414 arrives at approximately the same time as the LU wave 404 of FIG. 4, as the LU waves 404, 414 both must travel through entire thickness of the phantom, as this experiment was a transmissive arrangement, rather than a reflective arrangement, with respect to the LU waves.

The plot 420 of FIG. 6 shows the detected waves for the simulated artery that is a 13 mm depth. The first PA wave 422 arrives at approximately 6 microseconds, followed by an LU wave 414 at approximately 28 microseconds. Again, the PA wave 422 arrives sooner than the equivalent PA waves 402, 412 because the distance traveled by the PA wave 422 is shorter than either of waves 402, 412. The LU wave 424 arrives at approximately the same time as the LU waves 404, 414 as the LU wave 424 also traverses the entire thickness of the phantom.

FIG. 7 is a perspective image of an exemplary imaging system 101 according to certain aspects of the present disclosure. A laser source 110 is positioned adjacent to a laser detector 130 and above the skin 25 of a patient. The system 101 has an illumination/scan pattern 501 wherein the laser source 110 emits a beam 112 of laser light at a particular frequency and illuminates an elliptical area 511 on the skin 25 and the laser detector 130 scans a rectangular scan area 521 that is adjacent but non-overlapping with the illumination area 511. The laser detector 130 is shown as performing a scan using a scanning beam 132 over a solid angle 134 but this is only illustrative and the laser detector may detect and measure the surface within the scan area 521 using any non-contacting measurement system, such as an interferometer, as generally known to those of skill in the art. In certain embodiments, a reflective media, for example a reflective tape, oil, or gel, may be applied to the skin 25 in the scan area 521 to aid in signal detection.

In certain embodiments, the beam 112 may be provided at an angle to the skin 25 so that while the illumination area 511 does not overlap the scanned area 521, the beam 112 will illuminate subdermal structures, e.g. arteries, in a region directly below the scanned area 521. In certain embodiments, the beam 112 may be provided as a pulsed beam with a series of pulses at determined frequency. In certain embodiments, the pulses may have durations in the range of 1-1000 nanoseconds. In certain embodiments, the pulses may have durations in the range of 5-100 nanoseconds. In certain embodiments, the pulses may have durations in the range of 10-50 nanoseconds. In certain embodiments, the pulses may be provided at a frequency in the range of 1-100 Hz. In certain embodiments, the pulses may be provided at a frequency in the range of 5-20 Hz. In certain embodiments, the beam 112 may be a series of 15 nanosecond pulses provided at a repetition rate of 11 Hz. In certain embodiments, the laser detector 130 may operate continuously while the laser source 110 provides a pulsed beam 112 while, in other embodiments, the laser detector 130 may operate only between the pulses of the pulsed beam 112.

FIGS. 8-11 depict example illumination and scan areas according to certain aspects of the present disclosure. FIG. 8 depicts, as seen from directly over an area of skin 25, an illumination/scan pattern 502 wherein a rectangular illumination area 512 is adjacent to and smaller than a scan area 522. FIG. 9 depicts an illumination/scan pattern 503 wherein a circular illumination area 513 disposed between two rectangular scan areas 523A, 523B, while partially overlapping the scan area 523B. FIG. 10 depicts an illumination/scan pattern 504 wherein a circular illumination area 514 is partially surrounded by an L-shaped scan area 524. FIG. 11 depicts an illumination/scan pattern 505 wherein a rectangular illumination area 515 is surrounded by a scan area 525. Other combinations of sizes, shapes, and degree of overlap of the illumination and scan areas will be apparent to those of skill in the art.

The disclosed examples of a non-contact imaging system may provide an improved ability to generate two-dimensional or three-dimensional images of internal structures, particularly arteries, to aid in diagnosis and treatment of a patient. The separation of the illumination and scan areas may provide an increased field-of-view, increased resolution or noise reduction in the generation of such images, and greater flexibility is the use of the system, and the non-contact aspect of the system may improve the usability, for example in treatment planning for a surgical procedure or catheter intervention.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

What is claimed is:

1. An imaging system comprising:
a light source configured to project a beam of light within a determined range of wavelengths onto an illumination area of a patient's skin; and
a detector configured to detect acoustic waves within a scan area of the patient's skin without contacting the patient's skin, wherein the scan area does not overlap the illumination area.

2. The imaging system of claim 1, wherein the beam of light is coherent.

3. The imaging system of claim 1, wherein the beam of light has a wavelength that is in the range of 500-2000 nanometers.

4. The imaging system of claim 3, wherein the beam of light has a wavelength that is in the range of 1000-1200 nanometers.

5. The imaging system of claim 1, wherein the beam of light is provided as a series of pulses having a pulse duration and a pulse frequency.

6. The imaging system of claim 5, wherein the pulse duration is in the range of 5-100 nanoseconds.

7. The imaging system of claim 5, wherein the pulse frequency is in the range of 5-20 Hz.

8. The imaging system of claim 1, wherein the beam of light is directed at an angle of less than or equal to 90° to the skin.

9. The imaging system of claim 1, further comprising a reference zone defined by the scan area projected perpendicular to the skin, wherein the beam of light is directed at an angle to the skin such that the beam of light intersects the reference zone at a determined depth.

10. The imaging system of claim 9, wherein the depth is in the range of 0-60 mm.

11. The imaging system of claim 1, wherein the illumination area is elliptical.

12. The imaging system of claim 1, wherein the illumination area is rectangular.

13. The imaging system of claim 1, wherein the detector emits a sensing beam of light and detects the reflection of the sensing beam from the patient's skin.

14. The imaging system of claim 1, wherein the detector comprises an interferometer.

15. A method of creating an image of a structure below a patient's skin, the method comprising the steps of:
illuminating an illumination area of the patient's skin with a pulse of light;
detecting the arrival of acoustic waves at the patient's skin within a scan area that does not overlap the illumination area; and
analyzing the detected acoustic waves to create an image of the structure.

16. The method of claim 15, wherein the step of illuminating an illumination area comprises the steps of:
illuminating the structure with a pulse of light that at least partially passes from the illumination area through tissue between the patient's skin and the structure, thereby creating photoacoustic (PA) waves that propagate toward the scan area; and
creating ultrasonic (LU) waves at the patient's skin that propagate inward to the structure and are reflected toward the scan area.

17. The method of claim 15, wherein the structure is an artery.

18. The method of claim 15, wherein the structure is a plaque deposit.

19. The method of claim 15, wherein the pulse of light is directed along an illumination axis that passes through a reference zone defined by the scan area projected perpendicular to the skin at a determined depth.

20. The method of claim 19, wherein the depth is in the range of 0-60 mm.
